(19) United States
(12) Patent Application Publication
(10) Pub. No.: US 2018/0286499 A1
(43) Pub. Date: Oct. 4, 2018

(54) CONTEXT-AWARE INFORMATION TOOLTIPS FOR PERSONAL HEALTH RECORDS

(71) Applicant: KONINKLIJKE PHILIPS N.V., EINDHOVEN (NL)

(72) Inventors: Bella Fadida-Specktor, Haifa (IL); Shlomy Reinstein, Nesher (IL); Maria Kaplinsky, Haifa (IL); Orit Samuelly, Haifa (IL)

(21) Appl. No.: 15/754,382

(22) PCT Filed: Aug. 9, 2016
(86) PCT No.: PCT/IB2016/054792

§ 371 Date: Feb. 22, 2018

Related U.S. Application Data
(60) Provisional application No. 62/210,046, filed on Aug. 26, 2015.

(51) Int. Cl. G16H 10/60; G16H 50/20; G16H 15/00

(52) U.S. Cl. CPC G16H 10/60; G16H 15/00; G16H 50/20

(57) ABSTRACT

A personal health record system (10) for a patient includes a medical terms recognition unit (20), a personalized term association unit (22) and a term report unit (30). The medical terms recognition unit (20) receives a document (14) into a personal health record (12) of the patient, identifies medical terms within the document (14) and associates at least one identified medical term with one of a medical complaint, a medical history, a physical examination, a medical treatment, a medical diagnosis, a medical condition or a medical test based on a medical knowledge model (18). The personalized term association unit (22) associates the at least one identified medical term with at least one attribute specific to the patient (64). The term report unit (30) displays on a display device (32) with the document the at least one attribute specific to the patient and with an explanation of the at least one attribute associated with the at least one identified medical term with the at least one identified medical term.

### FIELD OF THE INVENTION

The following generally relates to patient access of health records with specific application to electronic personal health records.

### BACKGROUND OF THE INVENTION

Healthcare organizations maintain health records of patients which visit each healthcare organization for use by healthcare practitioners providing services at a respective healthcare organization. The health records can be stored as paper and/or electronically as an electronic medical record (EMR). EMRs include a record of patient complaints, patient history and demographic information, physical examination information, test results, diagnoses, treatments including orders and prescriptions, and the like. A health record can be represented as a series of documents, each document concerning a patient and prepared by one or more healthcare practitioners.

Trends in healthcare now see patients maintaining their own healthcare record as a personal health record (PHR). The PHR differs from the EMR in the scope and source of records. For example, a PHR can include documents received from healthcare practitioners at multiple healthcare organizations, where each healthcare organization maintains a separate record for the same patient independently of other healthcare organizations.

The PHR also differs from the EMR in access. A healthcare professional typically accesses the EMR and interprets the information contained within based on professional training and expertise. A patient typically accesses the PHR and typically uses Internet searches to interpret individual medical terms contained within a particular document. Internet searches do not include considerations of information about the patient, e.g. context-aware, which may aide the patient in understanding the voluminous definitions received in the search.

Furthermore, with the patient receiving and updating the PHR, issues which can arise based on different documents may go unnoticed. For example, a patient receives a first report from a first practitioner that identifies a condition, a diagnosis, or a test result may be impacted by a prescription from another practitioner for another condition. The other practitioner may not have seen or be aware of the other condition, diagnosis or test result, and the patient is not trained to recognize a potential problem with a prescription. It is also increasingly difficult for even healthcare practitioners to be aware of relevant changes across multiple specialties and pharmaceuticals.

### SUMMARY OF THE INVENTION

Aspects described herein address the above-referenced problems and others.

The following describes a personal health record (PHR) system for a patient and a method of accessing the PHR record, which provide a tooltip display according to medical terms in documents received into the PHR record. The tooltips display can include a clinical collision and/or a personalized explanatory information, which include at least one attribute specific to the patient.

In one aspect, a personal health record system for a patient includes a medical terms recognition unit, a personalized term association unit and a term report unit. The medical terms recognition unit receives a document into a personal health record of the patient, identifies medical terms within the document and associates at least one identified medical term with one of a medical complaint, a medical history, a physical examination, a medical treatment, a medical diagnosis, a medical condition or a medical test based on a medical knowledge model. The personalized term association unit associates the at least one identified medical term with at least one attribute specific to the patient. The term report unit displays on a display device with the document the at least one attribute specific to the patient and with an explanation of the at least one attribute associated with the at least one identified medical term with the at least one identified medical term.

In another aspect, a method of personal health records access includes receiving a document into a personal health record of the patient. At least one identified medical term within the document is associated with one of a medical complaint, a medical history, a physical examination, a medical treatment, a medical diagnosis, a medical condition or a medical test based on a medical knowledge model. The at least one identified medical term is associated with at least one attribute specific to the patient. The at least one attribute specific to the patient and an explanation of the at least one attribute associated with the at least one identified medical term is displayed on a display device with the at least one identified medical term in the document.

In another aspect, a personal health record system for a patient includes a medical terms recognition unit, a personalized term association unit and a term report unit. The medical terms recognition unit receives a document into a personal health record of the patient, identifies medical terms within the document and associate at least one identified medical term with one of a medical complaint, a medical history, a physical examination, a medical treatment, a medical diagnosis, a medical condition or a medical test based on a medical knowledge model. The personalized term association unit associates the at least one identified medical term with at least one attribute specific to the patient and generate at least one of a clinical collision or a personalized explanatory association. The term report unit displays on a display device the at least one identified medical term within the document and co-located with the at least one identified medical term the generated at least one of the clinical collision or the personalized explanatory association.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an embodiment of a context-aware information tooltips for personal health records system.

FIG. 2 illustrates an exemplary tooltips display with a personalized explanatory information and a clinical collision.

FIG. 3 illustrates an exemplary tooltips display with another clinical collision.

FIG. 4 illustrates an exemplary tooltips display with another personalized explanatory information.

FIG. 5 flowcharts an embodiment of generating context-aware information tooltips for personal health records.

FIG. 6 flowcharts an embodiment of associating personalized terms.

### DETAILED DESCRIPTION OF EMBODIMENTS

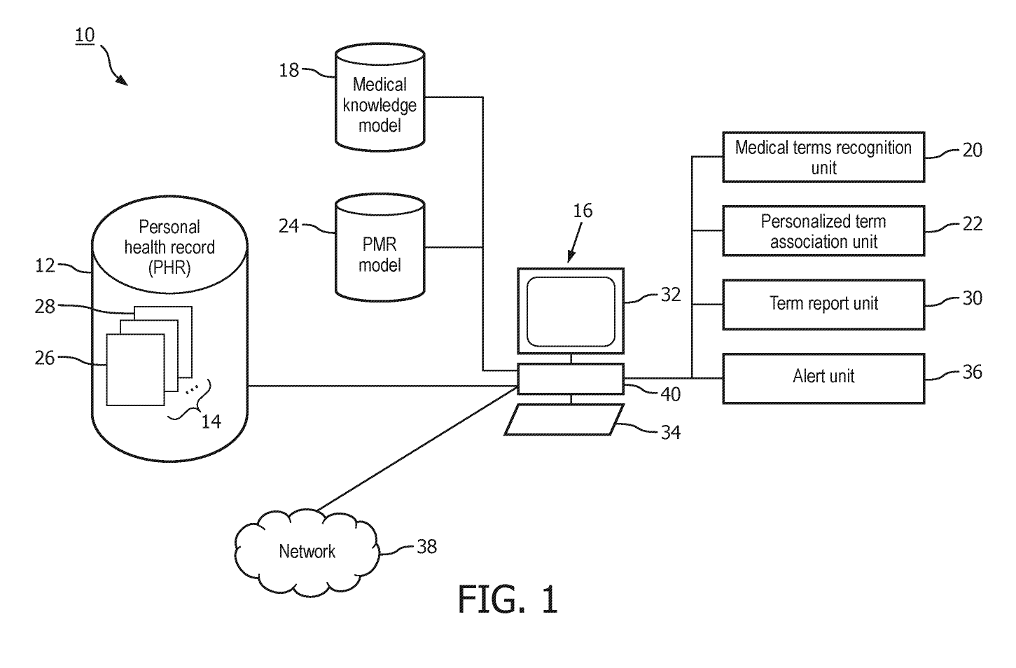

Initially referring to FIG. 1, an embodiment of a context-aware information tooltips for personal health record (PHR) system 10 is schematically illustrated. A PHR 12 includes documents 14 received from healthcare providers for a patient. For example, a prescription is received from a first healthcare provider for the patient, a test report is received from a second healthcare provider for the patient, and a referral for an imaging procedure is received from a third healthcare provider for the patient. Each document or report is received into the PHR by the patient. The PHR can include data which is entered by the patient, such as demographic data, profile data, patient history include family history and gene history, patient status, or data manually entered from the one or more documents 14. The PHR is stored in a computer memory, such as local storage, portable storage, cloud storage, Internet storage, and the like. For example, a patient can carry the PHR on a Universal Serial Bus (USB) flash drive which is accessible by one or more computing devices 16.

A medical knowledge model 18 includes medical terms, descriptions, and associations between medical terms and between medical terms and patient characteristics. The medical terms include diseases, symptoms, drugs, allergies, scans, tests, medical procedures, and the like. The medical knowledge model 18 can include mappings to one or more public medical ontologies, such as Systematized Nomenclature of Medicine (SNOMED), and the like. Patient characteristics can include demographic information, gene information, and family history. Associations can include indications, contraindications, normal conditions, and abnormal conditions.

A medical terms recognition unit 20 identifies relevant medical terms and respective locations from a received document 14. The medical terms recognition unit 20 can convert document images to text, e.g. perform optical character recognition (OCR) of text in a document image. The medical terms recognition unit 20 uses natural language processing known in the art to identify medical terms used in the document 14. The medical terms recognition 20 matches the identified medical terms from the document 14 with medical terms in the medical knowledge model 18.

The matching can include non-exact matching based on a probability that two terms and/or associations are the same. For example, drug names in a text document can include trade names, misspellings, and/or abbreviations. A probability can be assigned that a drug name X in a document 14 is a probable match to a drug name Y in the medical knowledge model 18.

A personalized term association unit 22 iteratively constructs a personal medical record (PMR) model 24 specific to the patient and identifies personalized medical associations based on the identified relevant medical terms from a currently received document 26 and/or one or more prior documents 28. The identified personalized medical association can include a clinical collision and/or an explanatory association. The clinical collision is a personalized contraindicated or abnormal association, which includes at least one attribute specific to the patient. The explanatory association is a personalized explanation of a relevant medical term, which includes an explanation with patient attribute. The patient attribute can include a characteristic of the patient, such as a physical attribute, test result, diagnosis, and the like.

The PMR model 24 can include identified medical terms and document locations. The PMR model 24 includes associations between data specific to the patient based on the associations of like terms in the medical knowledge model 18. The PMR model 24 and the medical knowledge model are suitably embodied by non-transitory computer memory. The models can include computer organization structures, such as database structure and systems, data structures, file structures and file systems. The computer memory can be local or remote, centralized or distributed.

A term report unit 30 constructs indicators of the personalized associations and displays the indicators as overlays or embedded into the documents 14. The term report unit 30 displays the indicators, such as an icon, highlighting, and the like, overlaid or embedded into the document 14 as the document 14 is displayed on a display device 32. The display device 32 can be embodied as a computer monitor, body worn display device, smartphone display, projection device, and the like. The term report unit 30 displays the clinical collision and/or personalized explanatory association in response to an input from an input device 34, such as a keyboard, mouse, microphone, touch screen, and the like.

An alerts unit 36 sends a notice of an identified clinical collision. The notice can include a formatted email message and/or a text message delivered via a network 38 according to profile information in the PHR 12 and/or PMR model 24. The message can include a text of the clinical collision. The text can be secured according to known methods of secured message transmission, such as encryption, authentication, and the like. In one embodiment, the alert unit 36 sends the notice based on access to the computing device 16.

The medical terms recognition unit 20, the personalized term association unit 22, the term report unit 30, and the alert unit 36 comprise one or more processors 40 (e.g., a microprocessor, a central processing unit, digital processor, and the like) configured to executes at least one computer readable instruction stored in a computer readable storage medium, which excludes transitory medium and includes physical memory and/or other non-transitory medium. The processor 40 may also execute one or more computer readable instructions carried by a carrier wave, a signal or other transitory medium. The processor 40 can include local memory and/or distributed memory. The processor 40 can include hardware/software for wired and/or wireless communications. The processor 40 can comprise the computing device 16, such as a desktop computer, a server, a laptop, a mobile device, a body worn device, distributed devices, combinations and the like.

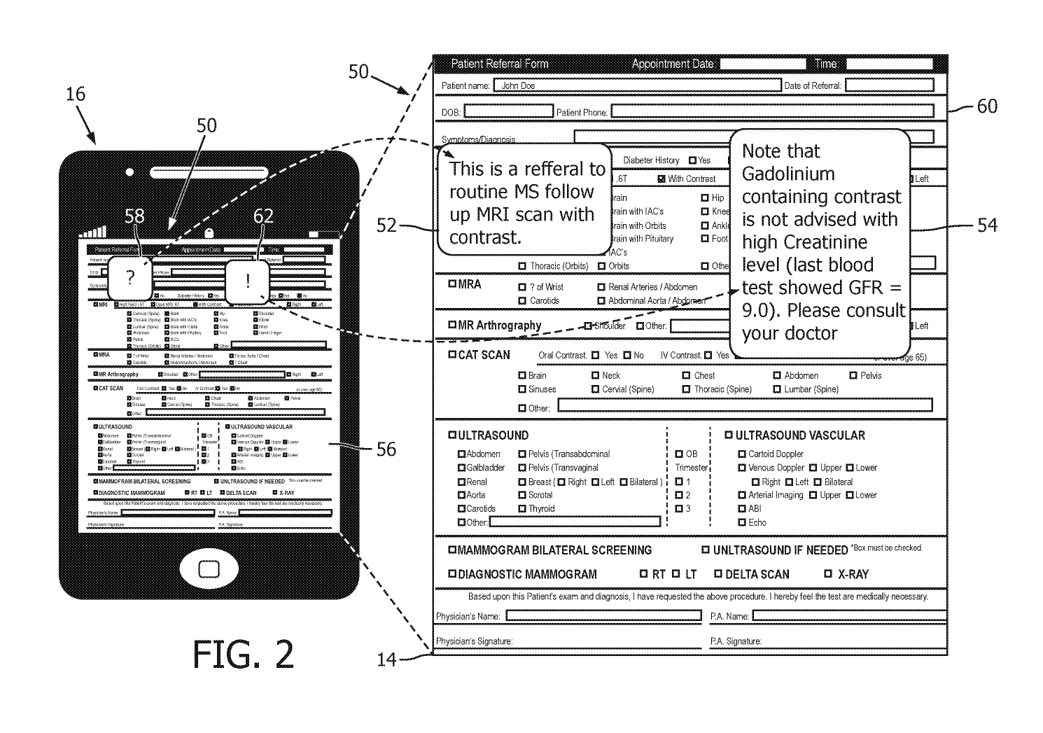

With reference to FIG. 2, illustrates an exemplary tooltips display 50 with a personalized explanatory information 52 and a clinical collision 54. The exemplary tooltips display 50 includes a document 14, which is a “patient referral form” that includes in a “symptoms/diagnosis” field “MS regular scan,” checked boxes corresponding to “MRI,” “with contrast,” “Brain,” “no cardiac condition,” “no diabetes history,” and “no contrast allergies.” The terms are recognized by the medical terms recognition unit 20 with probable matching or soft matching of the term MS as an abbreviation for multiple sclerosis in the medical knowledge model 18. The probable matching can include other information in the PMR model 24.

In a first exemplary tooltips display 56, a first icon 58 indicates an explanatory association is positioned near the term “MRI.” In response to an input, such as a touch co-located with the displayed first icon 58, a pop-up display of the personalized explanatory information 52 is displayed in a second exemplary tooltips display 60 as an overlay with the text “This is a referral to a routine MS follow-up MRI scan with contrast.” The text personalizes the association based on the PMR model 24 and the medical knowledge model 18 to indicate that it is “routine” and “follow up” based on associations of the medical knowledge model 18 and related to the MS based on the PMR model 24, which is specific to the patient.

In the first display 56, a second icon 62 positioned near the term “with contrast” indicates the clinical collision 54. In response to an input, a pop-up display of the clinical collision 54 is displayed as illustrated in the second display 60. The clinical collision includes the text “Note that Gadolinium containing contrast is not advised with Creatinine level (last blood test showed GFR=9.0) Please consult your doctor.” The PMR model 24 includes patient attributes identified from a prior document 28, which is a blood test. The test result includes Creatinine levels and a glomerular filtration rate (GFR) of 9.0. The medical knowledge model 18 associates the GFR with MRI scan and contrast agents, specifically gadolinium, which is contraindicated for patients with low GFRs (<30). The personalized term association unit 22 identifies the clinical collision based on the associated medical terms of “MRI” and “contrast” contraindicated for persons with low GFR when using gadolinium based contrast in the medical knowledge model 18 with the specific GFR=9.0 of the patient from the PMR model 24. The term report unit 30 constructs the two displays. The term report unit 30 constructs the first display with the icons indicative of the explanatory association and the clinical collision in an overlay on the displayed document 14. The term report unit 30 constructs the personalized text of the clinical collision 54 based on the clinical collision 54 generated by the personalized term association unit 22 and displays the text of the clinical collision 54 superimposed or overlaid on the displayed document 14 in the second display 60.

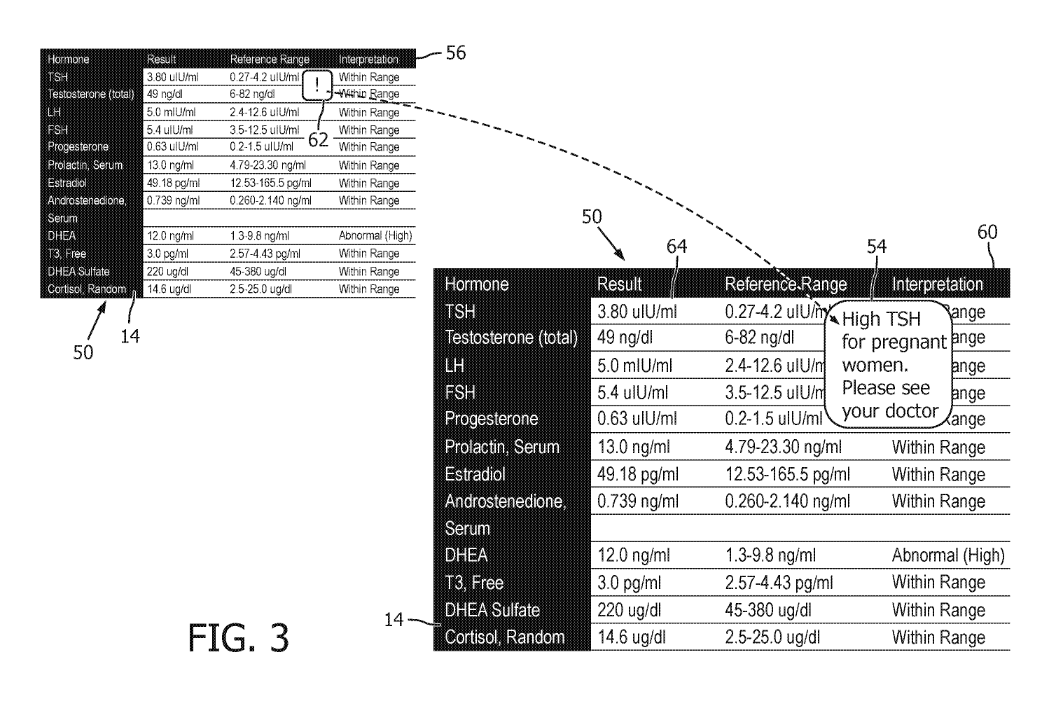

With reference to FIG. 3, an exemplary tooltips display 50 with another clinical collision 54 is illustrated. The tooltips display 50 includes the document 14, which is a blood test report. The blood test report includes a patient attribute (64) or a result for thyroid stimulating hormone (TSH) of 3.80 ulU/ml with a reference range of 0.27-4.2 ulU/ml. The result of TSH is indicated as within the reference range, e.g. normal for adults. The clinical collision indicator 62 is co-located on the line of the TSH result of the first display 56. In response to input, the text of the clinical collision 54 is displayed on a second display 60.

The PMR model 24 includes the patient attribute of a diagnosis that the patient is pregnant according to a prior document 28 or entry. The medical term unit 20 identifies the key terms “Hormone,” “TSH,” “Result,” and the values corresponding to the identified term “TSH” from the document 14. The personalized term association unit 22 generates the clinical collision 54 based on the medical knowledge model 18, which identifies a range of TSH for pregnant women (0.6-3.4) that is lower than non-pregnant adults, and that the patient is a pregnant woman from the PMR model 24 with a TSH value of 3.8, which is above a normal range for pregnant women. The clinical collision 54 includes a text “High TSH for pregnant women. Please see your doctor.” The clinical collision 54 includes the information that the patient is a pregnant woman from the PMR model 24, which is specific to the patient and associated with the TSH term from the document 14.

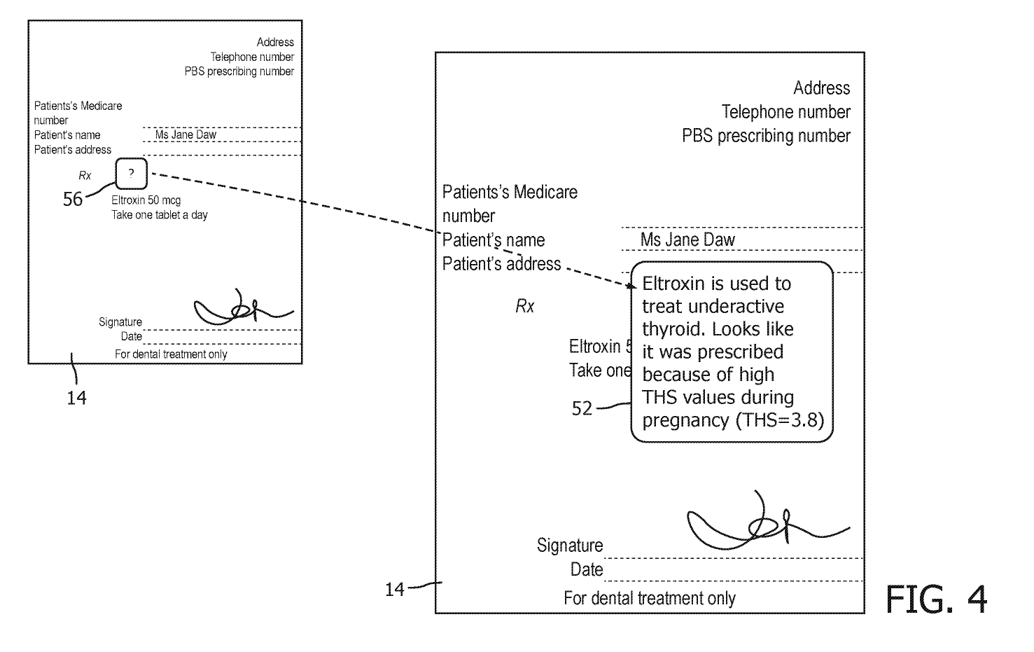

With reference to FIG. 4, an exemplary tooltips display 50 with another personalized explanatory information 52 is illustrated. The exemplary tooltips display 50 includes a document 14, which is a prescription. The medical terms recognition unit 20 identifies and associates the terms “Rx” and “Eltroxin” from the document 14 and the medical knowledge model 18 as a prescription for the drug Eltroxin, which is used to treat an underactive thyroid. The personalized term association unit 22 associates the prescription as a treatment and the drug Eltroxin for the pregnant patient discussed in reference to FIG. 3. The association includes information from the PMR model 24, which includes the patient attribute of a high TSH value of 3.80 from a blood test, and that the patient is pregnant, e.g. patient attribute diagnosis of pregnant, to generate the personalized explanatory information.

The term report unit 30 displays the second exemplary tooltips display 60 in response to an input, and the text of the personalized explanatory information 52 includes “Eltroxin is used to treat underactive thyroid. Looks like it was prescribed because of high TSH values during pregnancy (TSH=3.8).” The text includes personalized associations of the prescribed, e.g. treatment, Eltroxin, e.g. drug name, with patient attributes or patient specific information of the TSH value of 3.8, e.g. test and test result value, and the pregnancy, e.g. diagnosis, from the PMR model 24. The personalized explanatory information 52 and indicator 58 of personalized explanatory information are displayed co-located with the medical term “Eltroxin” on the respective displays.

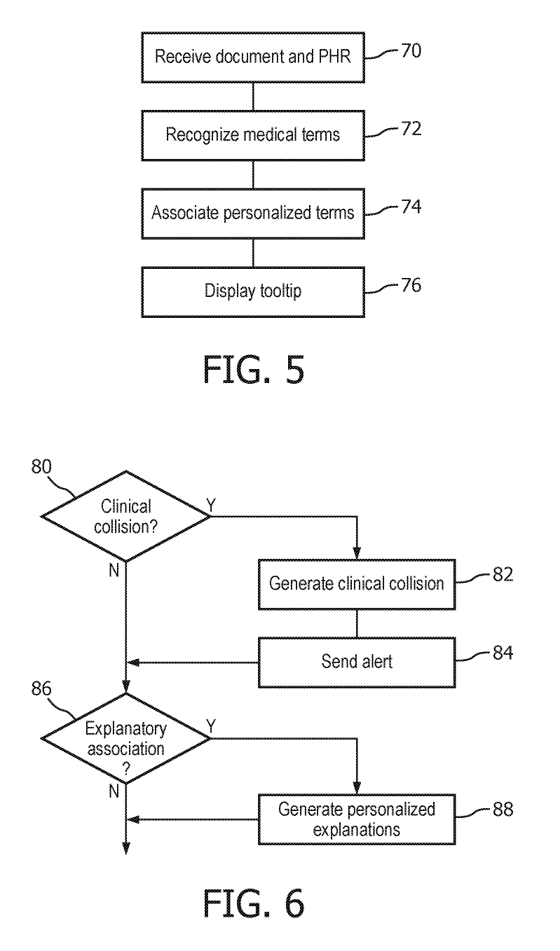

With reference to FIG. 5, an embodiment of generating context-aware information tooltips for personal health records (PHR) is flowcharted.

At 70 one or more documents 14 are received, which are included in the PHR 12. The documents can be received by electronic transfer, manual entry, or by reference. For example, the computing device 16 receives an electronic transfer of a document 14 as an email attachment from a healthcare provider. In another embodiment, the patient enters a universal resource locator (URL) of the document 14, which by reference retrieves the document. A PMR model 24 can be received if it exists.

At 72 medical terms are identified in the received document 14. Identified medical terms are associated with medical terms in the medical knowledge model 18. Identified medical terms can include a location of the medical term in the document 14. For example, drug names are associated with normalized drug names, treatments, symptoms, diagnoses, indications, contraindications, and the like. Identifying can include converting the document to a machine readable format, e.g. OCR. Identifying can include natural language processing to associate context with terms. Associating can include recognizing the type of document 14, such as a test result, prescription, diagnosis, test order, and the like. The associating can include natural language processing, which provides context to the identified medical term.

Personalized medical terms are generated at 74. The personalized medical terms can include clinical collisions 54 and/or personalized explanatory information 52. In one embodiment, the personalized medical terms can include a default patient oriented explanation, e.g. non-technical general explanation of the identified medical term. The PMR model 24 is updated with the associated personalized medical term. The associated personalized medical term includes the patient attribute and the associated medical term from the medical knowledge model 18. The PMR model 24 updates can include values and/or qualifiers associated with the personalized medical term.

At 76 the personalized medical terms are displayed co-located with the identified medical term on a display of the received document 14. The displayed personalized medical terms can include an indicator, which in response to an input selecting the indicator displays the personalized medical term.

With reference to FIG. 6, an embodiment of associating personalized terms is flowcharted. At 80, associating personalized medical terms include identifying clinical collisions 54 based on the identified medical terms, the medical knowledge model 18, and patient attributes from the PMR model 24. For example, if the identified medical term of a treatment or a test result with a value from the document 14 and/or associated term in the PMR model 24, which is contraindicated and/or abnormal based on the medical knowledge model 18, a clinical collision is identified with the identified medical term of the document 14.

In response to the identified clinical collision, the clinical collision text is generated and associated with the identified medical term at 82 and stored in the computer memory associated with the document 14. In one embodiment the text of the generated clinical collisions 54 is stored in the PMR model 24. In another embodiment, the text of the generated clinical collision is stored with the PHR 12. In another embodiment, a reference are stored with the PHR 12, such as pointers to the indicators with locations within the document, and the text of the clinical collision 54 is dynamically generated based on the reference from the PMR model 24 and/or medical knowledge model 18. The text includes patient specific information includes at least one association from the PMR model 24, such as between one of a complaint, history, physical examination, treatment, diagnosis, condition, test, or the like, which is contraindicated or abnormal based on the medical knowledge model 18.

An alert can be sent at 84. The alert can be sent to one or more recipients. The recipient can include the computing device 16 controlled by the patient and/or a computing device of a healthcare provider. The alert can be sent via a data and/or cellular network.

At 86, associating personalized medical terms include identifying personalized explanatory information 52 based on the identified medical terms, the medical knowledge model 18, and the PMR model 24. For example, if the identified medical term of a complaint, history, physical examination, treatment, diagnosis, condition, test, or the like from the document 14 and/or associated term in the PMR model 24, which is normal and/or indicated based on the medical knowledge model 18, a personalized explanatory information 52 is identified with the identified medical term of the document 14. In one embodiment, identified personalized explanatory information 52 can be limited to key medical terms based on an indicator or flag in the medical knowledge model 18.

In response to the identified personalized explanatory information, At 88, the personalized explanatory information 52 is generated and stored in the computer memory associated with the document 14. In one embodiment the text of the generated personalized explanatory information 52 is stored in the PMR model 24. In another embodiment, the text of the generated personalized explanatory information is stored with the PHR 12. In another embodiment, a reference are stored with the PHR 12, such as pointers to the indicators with locations within the document, and the text of the personalized explanatory information 52 is dynamically generated based on the reference from the PMR model 24 and/or medical knowledge model 18. The text includes patient specific information includes at least one association from the PMR model 24, such as between one of a complaint, history, physical examination, treatment, diagnosis, condition, test, or the like, which is indicated or normal based on the medical knowledge model 18.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A personal health record system for a patient, comprising:
a medical terms recognition unit which includes one or more processors configured to receive a document into a personal health record of the patient, identify medical terms within the document and associate at least one identified medical term with one of a medical complaint, a medical history, a physical examination, a medical treatment, a medical diagnosis, a medical condition or a medical test based on a medical knowledge model;
a personalized term association unit which includes the one or more processors configured to associate the at least one identified medical term with at least one attribute specific to the patient; and
a term report unit which includes the one or more processors configured to display on a display device with the document the at least one attribute specific to the patient and with an explanation of the at least one attribute associated with the at least one identified medical term with the at least one identified medical term.

2. The system according to claim 1, wherein the personalized term association unit identifies a clinical collision corresponding to the associated at least one identified medical term with the at least one attribute specific to the patient based on associations within the medical knowledge model which are at least one of contraindicated or abnormal according to the at least one attribute specific to the patient.

3. The system according to claim 1, wherein the personalized term association unit identifies a personalized explanatory information corresponding to the associated at least one identified medical term with the at least one attribute specific to the patient based on associations within the medical knowledge model which are at least one of indicated or normal according to the at least one attribute specific to the patient.

4. The system according to claim 1, further including:
a personal medical record model configured to store the associated at least one the at least one identified medical term with the at least one attribute specific to the patient and locations of the at least one identified medical term within the document.

5. The system according to claim 1, wherein the term report unit is configured to initially display an indication of associated at least one identified medical term with the at least one attribute specific to the patient co-located with the displayed one identified medical term.

6. The system according to claim 5, wherein the indication includes at least one of an icon embedded within the document or an icon displayed as an overlay.

7. The system according to 2, further including:
an alert unit which includes the one or more processors configured to send an alert of the clinical collision to a computing device of the patient.

8. The system according to claim 7, wherein the alert includes at least one of an email message or a text message, and the alert includes a personalized explanation of the clinical collision.

9. The system according to claim 1, wherein the at least one attribute specific to the patient a diagnosis or a test result.

10. The system according to claim 1, wherein the at least one attribute specific to the patient includes at least one patient characteristic.

11. A method of personal health records access, comprising:
receiving a document into a personal health record of the patient;
associating at least one identified medical term within the document with one of a medical complaint, a medical history, a physical examination, a medical treatment, a medical diagnosis, a medical condition or a medical test based on a medical knowledge model;
associating the at least one identified medical term with at least one attribute specific to the patient; and
displaying on a display device with the at least one identified medical term in the document the at least one attribute specific to the patient and an explanation of the at least one attribute associated with the at least one identified medical term.

12. The method according to claim 11, wherein associating includes:
identifying a clinical collision corresponding to the associated at least one identified medical term with the at least one attribute specific to the patient based on associations within the medical knowledge model which are at least one of contraindicated or abnormal according to the at least one attribute specific to the patient.

13. The method according to claim 11, wherein associating includes:
identifying a personalized explanatory information corresponding to the associated at least one identified medical term with the at least one attribute specific to the patient based on associations within the medical knowledge model which are at least one of indicated or normal according to the at least one attribute specific to the patient.

14. The method according to claim 11, wherein associating includes:
storing the associated at least one the at least one identified medical term with the at least one attribute specific to the patient.

15. The method according to claim 11, wherein includes:
initially displaying an indication of associated at least one identified medical term with the at least one attribute specific to the patient co-located with the displayed one identified medical term.

16. (canceled)

17. (canceled)

18. (canceled)

19. (canceled)

20. (canceled)
